(19) United States
(12) Patent Application Publication
(10) Pub. No.: US 2010/0301132 A1
(43) Pub. Date: Dec. 2, 2010

(54) FRAGRANCING DEVICE AND METHOD OF MAKING THE SAME

(76) Inventors: Holly Wagstaff-Bellomo, Denver, CO (US); Michael Tate, Denver, CO (US)

Correspondence Address: SHERIDAN ROSS PC, 1560 BROADWAY, SUITE 1200, DENVER, CO, 80202, US

(21) Appl. No.: 12/769,942

(22) Filed: Apr. 29, 2010

Related U.S. Application Data
(60) Provisional application No. 61/173,810, filed on Apr. 29, 2009.

(51) Int. Cl. A61L 9/04; B23P 17/04

(52) U.S. Cl. 239/36; 29/428

(57) ABSTRACT

The invention relates generally to a device for dispersing one or more fragrant materials, more particularly to a device in the form of an amulet for dispersing one or more fragrant materials.

### CROSS REFERENCE TO RELATED APPLLICATION

The present application claims the benefit of U.S. Provisional Application Ser. No. 61/173,810 filed Apr. 29, 2009, entitled “Fragancing Device and Method of Making the Same” which is incorporated herein by this reference in its entirety.

### FIELD OF THE INVENTION

The invention relates generally to a device for dispersing one or more fragrant materials, more particularly to a device in the form of an amulet for dispersing one or more fragrant materials.

### BACKGROUND OF THE INVENTION

The human sense of smell is a powerful sense interconnected to human memory, psyche, and physiology. Certain aromas can recall a memory or invoke an emotional response. Fragrant substances known as pheremones can bypass the human consciousness to trigger specific physiological behaviors. Aromatherapy is a form of homeopathy that relies on certain essential oils and/or fragrances to affect the human psychological and psychological response in order to treat certain human conditions.

### SUMMARY OF THE INVENTION

In one aspect of the present invention, the device is an amulet comprising a device body having a void and a dispersing material positioned adjacent to or within the void.

Various embodiments of the present invention are directed to a device and method for dispersing at least one fragrant material. Preferably, the at least one fragrant material has aromatherapy properties.

The dispersing material suitable for various embodiments can comprise any material that provides desired atributes in terms of being able to contain, retain and appropriately convey compounds, substances, liquids , and so forth having and/or meeting predetermined olfactory criteria. Thus, suitable dispersing material may include one or more of the following: a synthetic polymeric material, a natural polymeric material, cotton, ramin, wool, wood, or mixtures thereof. In one preferred embodiment, the dispersing material comprises wood. The wood can be selected from the group consisting of pine, cherry, oak, maple, walnut, ash, birch, hickory, teak, ebony, iron wood, padauk, blood wood, rosewood, bubinga, bucta, cannary wood, cocbuto, lacewood, purple wood, wenga, fir, beach, mesquie, merbau, purpleheart wood, cypress, spruce, sycamore, willow, yew, and mahogany. In an even more preferred embodiment, the material is mahogany, selected from the group consisting of South American mahogany, African mahogany, Khaya Ivorensis, Khaya nyassica, khaya senegalensis, Sengal mahogany, Khaya Anthotheca, Khaya Grandifoliola, Santos, Honduras mahogany, Ipé, and Jarrah.

The device body may comprise a natural or a synthetic mineral. In one preferred embodiment, the body comprises a stone, rock, or mineral, preferably having desirable aesthetic characteristics and/or indicative of an individual's birthstone, astrological sign, or notable feature. Preferably, the mineral is selected from the group consisting of: lapis, tigers eye, amazonite, speckled jade, amethyst, red jasper, opalite, oynx, and black agate. One or more surfaces of the device body can be polished or unpolished. In one preferred embodiment, one or more of the exterior surfaces of the device body are polished.

The device has a device width and a device height suitable for wearing by an individual, such as, around their neck and/or from an appendage, as for example from an ear, arm, nose, angle, wrist, and/or phalange. In a preferred embodiment, the device width is from about 6 mm to about 55 mm and the device height is from about 6 mm to from about 100 mm. In a more preferred embodiment, the device width is from about 10 mm to about 25 mm and the device height is from about 10 to about 60 mm.

The device can have one or more voids. A void width and a void depth, in one embodiment, is from about 2 mm to about 15 mm and the void depth is from about 2 mm to about 25 mm. In a preferred embodiment, the void width is from about 4 to about 10 mm and the void depth is from about 3 mm to about 10 mm.

In another embodiment, the device comprises a device body having a void, a bushing, and a dispersing material. The bushing is preferably positioned adjacent to the void and between the device body and the dispersing material. The bushing comprises one of a nylon bushing, a polymeric bushing, and a metallic bushing.

Preferably, the device is fashioned or formed into a portion of one or more of a necklace, an earring, an air fresher, a lapel pin, a hair-pin, a bookmark, a household decorative item, a personal accessory item, or a combination thereof.

Another aspect of the present invention is a process for making a device for dispersing one or more aromatic materials comprising: a) providing a device body; b) forming a void within the device body; c) providing a dispersing material; and d) positioning the dispersing material at least partially within the void.

In one embodiment the method further comprises: e) providing a bushing; and f) positioning the bushing at least partially in at least one of the voids prior to the positioning the dispersing material at least partially within or adjacent to one of the voids.

In another embodiment the method further comprises: g) providing a attachment element; and h) attaching the attachment element to the device body.

### BRIEF DESCRIPTION OF THE FIGURES

FIG. 1 depicts an elevation view of one embodiment of the present invention;

FIG. 2 depicts a cross-sectional view of one embodiment of the present invention.;

FIG. 3 depicts another embodiment of the present invention;

FIG. 4 depicts another embodiment of the present invention;

FIG. 5 depicts another embodiment of the present invention;

FIG. 6 depicts another embodiment of the present invention; and

FIG. 7 depicts yet another embodiment of the present invention.

### DETAILED DESCRIPTION OF THE INVENTION

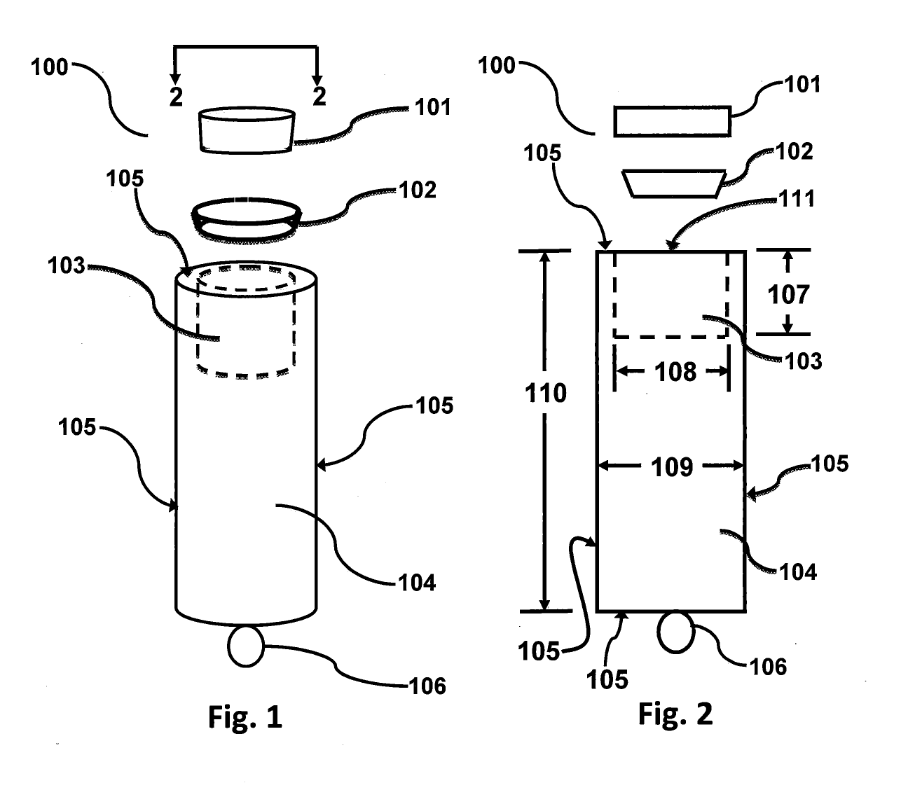

FIGS. 1 and 2 depict an embodiment of a device 100 of the present invention. The device is well suited for dispersing one or more fragrant materials. Preferably, the one or more fragrant materials have aromatherapy properties. Aromatherapy commonly means the use of volatile fragrant plant materials, may essential oils and/or aromatic compounds from plants for the purpose of affecting a person's mood and/or health. These fragrant plant materials are usually obtained by a distillation process that recover phytomolecules, which are may monoterpenes and sesquiterpenes, as well as other volatile substances, such as, but not limited to esters, aromatic compounds, non-terpene hydrocarbons, and sulfides. Non-limiting examples of suitable fragrant materials are disclosed in “Common Fragrance and Flavor Materials: Preparation, Properties and Uses, 5th edition” by H. Surburg and J. Panten, John Wiley & Sons, 2006 (ISBN-10 3-52731315-X), which is incorporated herein by this reference in it entirety.

Examples of suitable aromatherapy essential oils are, without limitation, those that comprise: Abelmoschus moschatus, Abies alba, Achillea millefolium, Amyris balsamifera, Anethum graveolens, Angelica archangelica, Aniba rosaeodora, Anthemis nobilis, Apis mellifera, Artemisia pallens, Backhousia citriodora, Boronia megastigma, Boswellia carterii, Cananga odorata, Cananga odorata var genuine, Callitris intratropica, Canarium luzonicum, Cedrus atlantica, Cinnamomum cassia, Cinnamomum zeylanicum, Cinnamosma fragrans, Citrus aurantifolia, Citrus aurantium (Neroli), Citrus aurantium (Bitter Orange), Citrus aurantium (Pettigrain), Citrus bergamia, Citrus junos, Citrus limon, Citrus paradisi, Citrus reticulata (Mandarin), Citrus reticulata (Tangerine), Citrus sinensis, Coffea Arabica, Commiphora myrrha, Coriandrum sativum, Cuminum cyminum, Cupressus sempervirens, Cymbopogon citratus, Cymbopogon martini, Cymbopogon nardus, Daucus carota, Dipterocarpus turbinatus, Elettaria cardamomum, Eucalyptus citriodora, Eucalyptus globulus, Eucalyptus radiata, Eugenia caryophyllata, Evernia prunastri, Ferula galbaniflua, Foeniculum vulgare, Helichrysum angustifolia, Hyssopus officinalis, Illicium verum, Jasminum officinalis, Juniperus communis, Juniperus virginiana, Laurus Nobilis, Lavendula abrialis, Lavandula hybrida, Lavendula officinalis, Leptospermum ericoides, Leptospermum petersonii, Leptospermum scoparium, Litsea cubeba, Matricaria chamomilla, Melaleuca alternifolia, Melaleuca leucadendron, Melaleuca quinquenervia, Melissa officinalis, Mentha Citrata, mentha piperita, Mentha piperita v. chocolat, Mentha spicata, Myristica fragrans, Myroxylon pereirae, Myrtus communis, Nardostachys jatamansi, Nepeta cataria, Nicotania tabacum, Ocimum basilicum, Ocimum gratissimum, Ocimum sanctum, Oreganum majorana, Oreganum vulgare, Pelargonium graveolens, Pelargonium roseum, Petroselinum sativum, Picea mariana, Pimenta racemosa, Pimento officinalis, Pimpinella anisum, Pinus sylvestris, Piper nigrum, Pogostemon cablin, Polianthes tuberosa, Ravensara aromatica, Rosa damascena, Rosmarinus officinalis, Salvia lavandulaefolia, Salvia officinalis, Salvia sclarea, Santalum album, Styrax tonkinensis, Tagetes minuta, Thymus vulgaris, Tilia vulgaris, Vanilla planifolia, Verbascum Thapsus, Vetiveria zizanoides, Viola odorata, Zingiber officinale, and mixtures thereof.

In one embodiment, the device 100 comprises a device body 104 having at least one void 103, exterior surface(s) 105, an optional bushing 102, a dispersing material 101, and an optional attachment element 106. The device 100 has a device width 109 and a device height 110.

The device body 104 may comprises a solid material, preferably a solid material that is chemically and physically stable that is not substantially damaged or degraded by one or more of the fragrant materials. That is, the device body is preferably not softened, embrittled, dissolved, swelled, solubilized, dissolved, dispersed, or otherwise damaged, and/or degraded by one or more of the fragrant materials. In one embodiment, the device body 104 comprises a mineral. The mineral can be natural, synthetic, or a combination thereof. Preferably, the mineral is stone. In one embodiment, the mineral comprises one of lapis, tigers eye, amazonite, speckled jade, amethyst, red jasper, opalite, oynx, quartz, glass, and black agate.

The device body 104 has at least one void 103. The void 103 has a void depth 107 and a void width 108. The void 103 can be positioned anywhere on the device body 104, preferably with at least one void aperture 111 positioned on at least one exterior surface 105. Preferably, the void depth 107 is less than the device height and the void width 108 is less than the device width 109. In one embodiment, the device width is from about 3 mm to about 55 mm and the device height is from about 3 mm to from about 100 mm. In a more preferred embodiment the device width is from about 10 mm to about 25 mm and the device height is from about 10 to about 60 mm. In one embodiment, the void width is from about 2 mm to about 15 mm and the void depth is from about 2 mm to about 25 mm. In a preferred embodiment, the void width is from about 4 to about 10 mm and the void depth is from about 3 mm to about 10 mm.

The dispersing material 101 is positioned adjacent to one or more of the void 103 and/or optional bushing 102. The one or more of the void 103 and optional bushing 102 substantially maintains the dispersing material 101 positioned on the device body 104 and secured thereto such that the dispersing material 101 is not may shaken, jarred, or jogged loose.

The dispersing material 101 is may shaped and/or deformed to mate with one or more of the void 103 and/or optional bushing 102. That is, dispersing material 101 is at least partially capable of being internally positioned in one or more of the void 103 and/or optional bushing 102.

The dispersing material 101, after being contacted with one or more fragrant materials, substantially retains the fragrant material for subsequent release for a desired period of time. Stated another way, the dispersing material 101 when contacted with one or more fragrant materials adsorbs the fragrance material(s) for gradual release into the surrounding environment. Release means can be employed to convey desired amounts of scent. Fragrant material is released from the dispersing material 101. In one embodiment, the dispersing material 101 is contacted with one or more fragrant materials prior to positioning the dispersing material 101 adjacent to one or more of the void 103 and/or optional bushing 102. In another embodiment, the dispersing material 101 is contacted with one or more fragrant materials after positioning the dispersing material 101 adjacent to one or more of the void 103 and/or optional bushing 102. The desired fragrant materials can be deposited by one of syringing, spraying, pouring, douching, pipetting or such.

One or more fragrant materials can be positioned in void 103 prior to the positioning the dispersing material 101 adjacent to one or more of the void 103 and/or optional bushing 102. In one particular embodiment, at least two voids are present. In yet another, at least three voids are present. The voids can be any desired shape, for example, to connote a sign, word, animal, number, symbol, character, graphic design, or such.

The dispersing material 101 can be any material capable of one of absorbing and/or adsorbing at least one fragrant material. The dispersing material 101 can comprise synthetic polymeric materials, natural polymeric materials, cottons, ramins, wools, woods, and mixtures thereof. In one preferred embodiment, the dispersing material 101 comprises wood. Preferably, the dispersing material 101 comprises one of pine, cherry, oak, maple, walnut, ash, birch, hickory, teak, ebony, iron wood, padauk, blood wood, rosewood, bubinga, bucta, cannary wood, cocbuto, lacewood, purple wood, wenga, fir, beach, mesquie, merbau, purpleheart wood, cypress, spruce, sycamore, willow, yew, mahogany, or a combination thereof. In an even more preferred embodiment, the dispersing material 101 is mahogany. A non-limiting example of suitable mahoganies are: South American mahogany, African mahogany, Khaya Ivorensis, Khaya nyassica, khaya senegalensis, Sengal mahogany, Khaya Anthotheca, Khaya Grandifoliola, Santos, Honduras mahogany, Ipé, and Jarrah.

Another aspect of the present invention is a method of making an amulet comprising: a) providing a mineral body; b) forming a void within the mineral body; (c) providing a dispersing material; and (d) positioning the dispersing material at least partially within the void. Another embodiment may includes (e) providing a bushing; and (f) positioning the bushing at least partially in the void, wherein the bushing is positioned between the mineral body and the dispersing material. An optional embodiment includes, (g) providing an attachment element; and (h) attaching the attachment element to the mineral body.

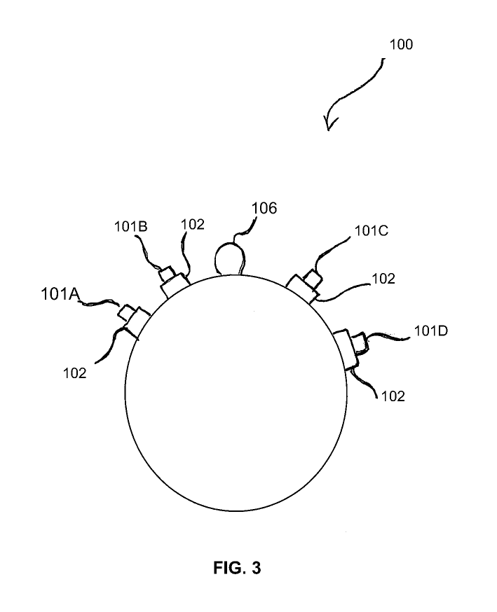

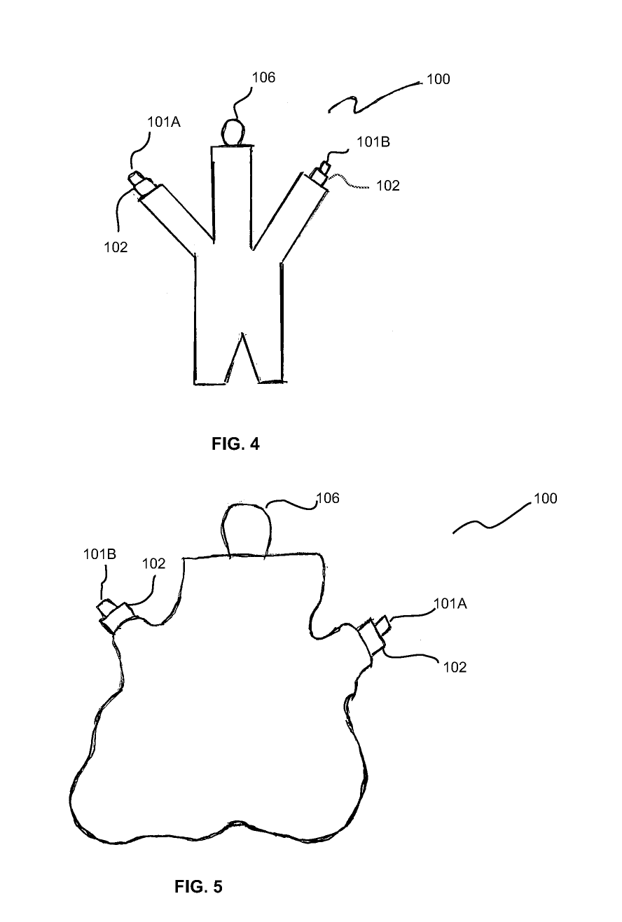

FIGS. 3-5 depict other, non-limiting, embodiments of the present invention. FIGS. 3-5 depict suitable alternative shapes of the device 100, for example, the device 100 can have a generally circular shape (FIG. 3), a shape resembling a figure and/or symbol (such as, a human, animal, mythical, astrological, religious, or other—FIG. 4), or an abstract shape (FIG. 5).

FIGS. 3-5 further depict embodiments of device 100 having a plurality of dispersing materials 101. The plurality of dispersing materials 101 can one or more of: a) disperse a greater quantity of fragrant material; b) disperse a plurality of differing fragrant materials; and c) provide a controlled release mechanism for one or more fragrant materials. Regarding the dispersing of a greater quantity of fragrant material, it can be appreciated that, ‘n’ number of dispersing materials can disperse about ‘n’ times as much fragrant material than a single dispersing material element (of comparable size, shape, and construction). Regarding the dispersing a plurality of differing fragrant materials, it can be appreciated that, that in one embodiment, dispersing element 101a can disperse fragrant material ‘a’, while dispersing element 101b can disperse a different fragrant material ‘b’ and so forth for dispersing elements 101c through 101z. It can be further appreciated that, the differing fragrant materials ‘a’ through ‘z’ can: 1) combine to synergistically provide an aromatherapy benefit or 2) individually provide different beneficial affects (such as, one fragrant material provides a relaxing affect while another fragrant material reduces blood pressure and yet another fragrant material stimulates the brain).

In yet another embodiment, the plurality of dispersing materials 101 comprises two sets of materials: a substantially dispersing set of materials and a substantially non-dispersing set of materials. The substantially dispersing materials disperse the fragrant material. The substantially non-dispersing materials seal the fragrant materials within the plurality of voids 103 for later use, that is, for dispersing at a later time by replacing the non-dispersing material with a dispersing material at the later time.

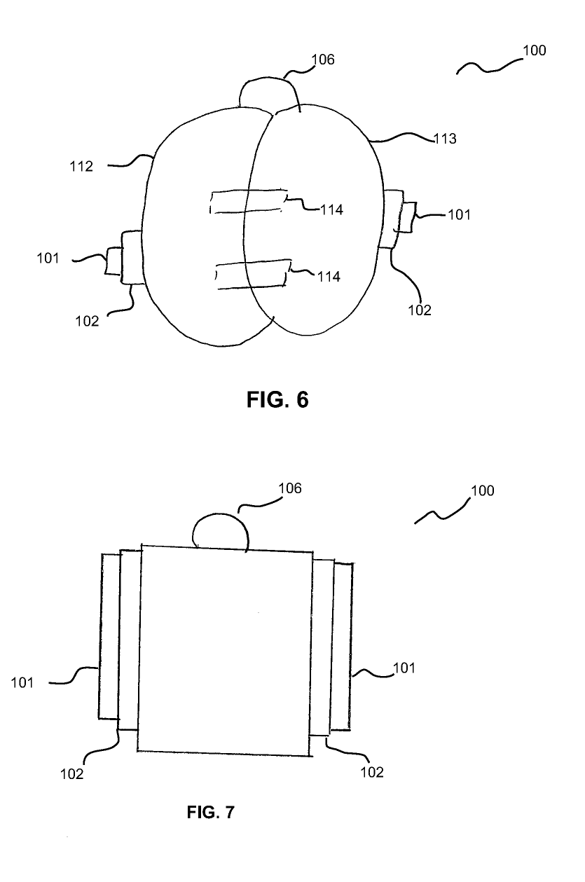

FIG. 6 depicts another embodiment. The device 100 of FIG. 6 comprises first 112 and second 113 elements adaptively interconnected by a plurality of components 114. The first 112 and second elements 113 can be provided separately and adaptively interconnected by the user. The first 112 and second 113 elements are separate and distinct. That is, the first 112 and second 113 elements can comprise different materials and have differing shapes.

FIG. 7 depicts yet another embodiment. The dispersing materials 101 are configured to provide a substantially large surface area for releasing the fragrant material. The large surface area dispersing material 101 can be utilized for fragrant materials that are one or more of: a) difficult to release into the environment and b) require a greater quantity released to affect an aromatherapy affect.

It can be further appreciated that in one embodiment the device 100 can be warmed by one of body, sun and/or environment. The warming of the device 100 can assist in the release of the fragrant material into the environment to further assist in providing an aromatherapy affect. In another embodiment, the device 100 is coated with a liquid crystalline material. The liquid crystalline material has different colors at different temperatures. For example, at one temperature the liquid crystalline material has a first color at a first temperature and a second color at a second color, the first and second color differ.

In still yet another embodiment, the attachment element 106, can substantially freely rotate. The free rotation of the attachment element 106 can provide for greater release of the fragrant material into the environment, compared to an attachment element 106 lacking free rotation.

While not being limited by example, typical uses of the amulet include without limitation an element of one or more of a necklace, an earring, an air fresher, a lapel pin, a hair pin, a bookmark, a household decorative item, a personal accessory item, or a combination thereof.

Components:
100 Device
101 Dispersing material
102 Bushing
103 Void
104 Device Body
105 Exterior Surface
106 Attachment Element
107 Void Depth
108 Void Width
109 Device Width
110 Device Height
111 Void aperture
112 First element
113 Second element
114 Component

What is claimed is:

1. An amulet comprising:
a device body having a void; and
a dispersing material position adjacent to the void.

2. The amulet of claim 1, further comprising a bushing, wherein the bushing is positioned adjacent to the void and wherein the bushing is positioned between the device body and the dispersing material.

3. The amulet of claim 1, further comprising an attachment element.

4. The amulet of claim 1, wherein the dispersing material comprises one of synthetic polymeric material, natural polymeric material, cotton, ramin, wool, wood, or mixtures thereof.

5. The amulet of claim 4, wherein in the wood is selection from the group consisting of pine, cherry, oak, maple, walnut, ash, birch, hickory, teak, ebony, iron wood, padauk, blood wood, rosewood, bubinga, bucta, cannary wood, cocbuto, lacewood, purple wood, wenga, fir, beach, mesquie, merbau, purpleheart wood, cypress, spruce, sycamore, willow, yew, and mahogany.

6. The amulet of claim 5, wherein the mahogany is selected from the group consisting of South American mahogany, African mahogany, Khaya Ivorensis, Khaya nyassica, khaya senegalensis, Sengal mahogany, Khaya Anthotheca, Khaya Grandifoliola, Santos, Honduras mahogany, Ipé, and Jarrah.

7. The amulet of claim 1, wherein the device body comprises one of lapis, tigers eye, amazonite, speckled jade, amethyst, red jasper, opalite, oynx, and black agate.

8. The amulet of claim 1, wherein the device body is polished.

9. The amulet claim 1, wherein the device body is unpolished.

10. The amulet of claim 2, wherein the bushing comprises one of nylon bushing, polymeric bushing, or metallic bushing.

11. The amulet of claim 1, wherein the amulet has a width and a length.

12. The amulet of claim 11, wherein the width is from about 6 mm to about 55 mm.

13. The amulet of claim 11, wherein the length is from about 6 mm to about 100 mm.

14. The amulet of claim 1, wherein the void has a diameter and a depth.

15. The amulet of claim 14, wherein the diameter is from about 5 mm to about 15 mm.

16. The amulet of claim 14, wherein the depth is from about 5 mm to about 25 mm.

17. A method of making an amulet comprising:
providing a mineral;
forming a void within the mineral;
providing a fragrance dispersing material; and
positioning the fragrance dispersing material at least partially within the void.

18. The method of claim 17, further comprising: providing a bushing; and
positioning the bushing at least partially in the void, wherein the bushing is positioned between the mineral and the fragrance dispersing material.

19. The method of claim 17, further comprising:
providing a attachment element; and
attaching the attachment element to the mineral.

20. The amulet of claim 1, wherein the amulet further comprises an element of one or more of a necklace, an earring, an air fresher, a lapel pin, a hair pin, a bookmark, a household decorative item, a personal accessory item, or a combination thereof.
